(19) United States
(12) Patent Application Publication
(10) Pub. No.: US 2018/0369518 A1
(43) Pub. Date: Dec. 27, 2018

(54) Piercing Device With Perforator, Heating Mechanism And Air Passages

(71) Applicant: Resolve Digital Health Inc., Toronto (CA)

(72) Inventor: Robert ADELSON, Vaughn (CA)

(73) Assignee: Resolve Digital Health Inc., Toronto (CA)

(21) Appl. No.: 16/020,544

(22) Filed: Jun. 27, 2018

Related U.S. Application Data
(60) Provisional application No. 62/525,532, filed on Jun. 27, 2017.

(51) Int. Cl. A61M 15/00

(52) U.S. Cl. CPC A61M 15/0036; A61M 15/0021; A61M 2205/36; A61M 2205/121; A61M 2205/123

(57) ABSTRACT

An aspect of the present disclosure provides a piercing device for use with an inhalation device, the piercing device including: at least one perforator; a plurality of passages defined along said at least one perforator; and at least one heating mechanism incorporated into said at least one perforator, wherein docking of said piercing device with the inhalation device holding a reservoir allows said at least one perforator to access content of the reservoir, and wherein activation of said at least one heating mechanism allows generation of vapors from the content of the reservoir to be carried through at least one of said plurality of passages to a user.

### TECHNICAL FIELD

The disclosure generally relates to the field of inhalation device. In particular, the present disclosure provides a piercing device to be used with an inhalation device to pierce the packaging material of a reservoir, to allow passage of ambient air through contents of the reservoir, to heat the contents of the reservoir to generate vapors and to convey the vapors to a user.

### BACKGROUND

Background description includes information that may be useful in understanding the present invention. It is not an admission that any of the information provided herein is prior art or relevant to the presently claimed invention, or that any publication specifically or implicitly referenced is prior art.

While smoking is generally not recommended due to the ill effects of smoke inhalation and the low efficiency in delivery the un-combusted bioactive agent, vaporization and inhalation of the vapors of drugs suffering from low bioavailability may present a viable solution to the problems associated with injection and ingestion thereof. A partial solution is provided by some vaporization techniques aimed at delivering inhaled vaporizable bioactive agents while avoiding the respiratory hazards of smoking. While the temperature at the center of a burning cigarette is 750-800° C., vaporization can be performed at any predetermined temperature, thereby allowing vapors of the bioactive agent to form below the combustion temperature, at which pyrolytic toxic compounds are generated. It has been shown that vaporization techniques reduce formation of carbon monoxide and highly carcinogenic compounds such as polynuclear aromatic hydrocarbons (PAHs), benzene and tar.

Oral or nasal delivery of a medicament using an inhalation device is a particularly attractive method of drug administration as these devices are relatively easy for a patient to use discreetly and in public. As well as delivering medicament to treat local diseases of the airway and other respiratory problems, they have more recently also been used to deliver drugs to the bloodstream via the lungs, thereby avoiding the need for hypodermic injections.

It is common for dry formulations to be pre-packaged so as to ensure each dose is hermetically sealed to protect each dose from the ingress of moisture and penetration of gases such as oxygen in addition to shielding the dose from light and UV radiation all of which can have a detrimental effect on the medicament and on the operation of an inhalation device used to deliver the medicament to a patient. Therefore, piercing the packaging material of a reservoir to access the pre-packaged single or multiple dose of a medicament, allowing ambient air to flow through the content of the reservoir, heating the content of the reservoir and allowing vapors to get conveyed to the user remains a significant challenge.

The present disclosure addresses these challenges along with mitigating one or more disadvantages associated with piercing devices known in the art.

### OBJECTS OF THE INVENTION

An object of the present disclosure is to overcome one or more limitations associated with conventional piercing device to be used with an inhalation device.

Another object of the present disclosure is to provide a piercing device that ensures easy penetration of the packaging material of the reservoir allowing access to the contents thereof.

Another object of the present disclosure is to provide a piercing device that allows passage of ambient air and vaporized contents of the reservoir without clogging.

Another object of the present disclosure is to provide a piercing device that is easy to use.

Other objects of the present disclosure will be apparent from the description of the invention herein below.

### SUMMARY OF INVENTION

The disclosure generally relates to the field of inhalation device. In particular, the present disclosure provides a piercing device to be used with an inhalation device to pierce the packaging material of a reservoir, to allow passage of ambient air through contents of the reservoir, to heat the contents of the reservoir to generate vapors and to convey the vapors to a user.

An aspect of the present disclosure provides a piercing device for use with an inhalation device, the piercing device comprising: at least one perforator; a plurality of passages defined along said at least one perforator; and at least one heating mechanism incorporated in said at least one perforator, wherein docking of said piercing device with the inhalation device holding a reservoir allows said at least one perforator to access content of the reservoir, and wherein activation of said at least one heating mechanism allows generation of vapors from the content of the reservoir to be carried through at least one of said plurality of passages to a user.

In an embodiment, said piercing device further comprises a mouth piece and a connector. In an embodiment, said connector defines a first chamber connected to said at least one of the plurality of passages to allow passage of the vapors generated from the content of the reservoir through said at least one of the plurality of passages. In an embodiment, said mouth piece defines a second chamber connected to said first chamber to allow passage of the vapors from said first chamber to said user. In an embodiment, said connector and said mouth piece define a contiguous chamber connected to said at least one of the plurality of passages to allow passage of the vapors generated from the content of the reservoir to the user. In an embodiment, said connector comprises at least one locking mechanism to detachably connect said perforator with said inhalation device. In an embodiment, the at least one heating mechanism comprises an induction heating mechanism. In an embodiment, said at least one inductive heating mechanism is selected from a group consisting of an inductive heating element and an inductive heating coil. In an embodiment, the inductive heating element is dispersed in said at least one perforator and wherein the inductive heating coil is arranged as part of the inhalation device.

In an embodiment, said at least one perforator is of pyramid shape defining one or a plurality of lateral surfaces and an apex. In an embodiment, said plurality of passages are defined at any or a combination of at least one of said one or a plurality of lateral surface and said apex, further wherein any or a combination of at least a part of said one or a plurality of lateral surfaces and said apex comes in contact with the content of the reservoir upon docking of said piercing device with the inhalation device. In an embodiment, said at least one perforator defines a plurality of elevations along said one or a plurality of lateral surfaces, further wherein at least one of said plurality of passages is defined along a surface of said plurality of elevations.

In an embodiment, said at least one perforator is of cylindrical shape with said plurality of passages defined at any or a combination of periphery of the at least one perforator and centre of the at least one perforator. In an embodiment, any or a combination of at least a part of said periphery of the at least one perforator and the centre of said at least one perforator comes in contact with the content of the reservoir upon docking of said piercing device with the inhalation device.

In an embodiment, said at least one perforator is of pyramid shape defining one or a plurality of lateral surfaces and a cylindrical protrusion at apex of said at least one perforator. In an embodiment, said plurality of passages are defined at: (a) any or a combination of periphery of said cylindrical protrusion and centre of said cylindrical protrusion and (b) said one or a plurality of lateral surfaces of said at least one perforator. In an embodiment, said at least one perforator defines a plurality of elevations along said one or a plurality of lateral surfaces defining a plurality of peaks and troughs. In an embodiment, at least one of said plurality of passages is defined at said plurality of troughs. In an embodiment, at least one of said plurality of passages is defined at said plurality of peaks. In an embodiment, at least one of said plurality of passages defined at said plurality of peaks allows passage of ambient air to the content of the reservoir, and wherein said at least one of said plurality of passages defined at said plurality of troughs allows passage of vapors generated from the content of the reservoir to the mouth piece.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1A illustrates an exemplary partially exposed front view of a piercing device docked to an inhalation device in accordance with an embodiment of the present disclosure.

FIG. 1B illustrates an exemplary perspective view of a piercing device in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Various terms as used herein are shown below. To the extent a term used in a claim is not defined below, it should be given the broadest definition persons in the pertinent art have given that term as reflected in printed publications and issued patents at the time of filing.

As used herein, a person skilled in the relevant art may generally understand the term “vaporization” or “vaporize” to generally mean the phase transition of an element, compound or composition etc. from a liquid or a solid (including moisture on the solid) phase to a vapor; with no burning, the absence of fire, smoke and the by-products of burning.

As used herein, a person skilled in the relevant art may generally understand the term “vapor” to generally mean a substance in the gas phase at a temperature lower than its critical point. It may also include one or more substances in a suspension of finely divided solid particles or liquid droplets in a gas, including aerosols, mists and the like.

As used herein, the term “pyramid” or “pyramid shape” denotes a three-dimensional shape that tapers from a base to a point called the apex and includes pyramid shape (with at least three sides/lateral surfaces that tapers from polygonal base to apex), conical shape (with one lateral surface that tapers smoothly from a flat base to apex) and like shapes with one or a plurality of lateral surfaces/sides tapering from base to apex.

The disclosure generally relates to the field of inhalation device. In particular, the present disclosure provides a piercing device to be used with an inhalation device to pierce the packaging material of a reservoir, to allow passage of ambient air through contents of the reservoir, to heat the contents of the reservoir to generate vapors and to convey the vapors to a user.

An aspect of the present disclosure provides a piercing device for use with an inhalation device, the piercing device comprising: at least one perforator; a plurality of passages defined along said at least one perforator; and at least one heating mechanism incorporated into said at least one perforator, wherein docking of said piercing device with the inhalation device holding a reservoir allows said at least one perforator to access content of the reservoir, and wherein activation of said at least one heating mechanism allows generation of vapors from the content of the reservoir to be carried through at least one of said plurality of passages to a user.

In an embodiment, said piercing device further comprises a mouth piece and a connector. In an embodiment, said connector defines a first chamber connected to said at least one of the plurality of passages to allow passage of the vapors generated from the content of the reservoir through said at least one of the plurality of passages. In an embodiment, said mouth piece defines a second chamber connected to said first chamber to allow passage of the vapors from said first chamber to said user. In an embodiment, said connector and said mouth piece define a contiguous chamber connected to said at least one of the plurality of passages to allow passage of the vapors generated from the content of the reservoir to the user.

In an embodiment, the device includes, in the connector, a vapor sensor for detecting and/or measuring composition and flow of air and/or vapor moving through the device. In an embodiment, the device includes, in the mouthpiece, a vapor sensor for detecting and/or measuring composition and flow of air and/or vapor moving through the device.

In an embodiment, the device includes, in the connector, a cooling coil for decreasing temperature of the vapor. In an embodiment, the device includes, in the mouthpiece, a cooling coil for decreasing temperature of the vapor. In an embodiment, the cooling coil is capable of reducing the temperature of the vapor to about 100 degrees Celsius. In alternate embodiments, vapor is cooled by adjusting the distance of travel between the chamber carrying the vapor and the mouthpiece.

In an embodiment, said connector comprises at least one locking mechanism to detachably connect said perforator with said inhalation device. In an embodiment, the locking mechanism includes a magnet. The locking mechanism can include any of the locking mechanism as known to a person skilled in the art including but not limited to a clip, a spring, a groove, a slit, a keyed opening, friction fitting and the like to serve its intended purpose of detachably connecting said piercing device with said inhalation device.

In an embodiment, the at least one heating mechanism comprises an induction heating mechanism. In an embodiment, said at least one inductive heating mechanism is selected from a group consisting of an inductive heating element and an inductive heating coil. In an embodiment, the inductive heating element is dispersed in said at least one perforator and the inductive heating coil is arranged as part of the inhalation device. Alternatively, at least a part of the perforator can be made of an electrically conductive material so as to serve as an inductive heating element. Alternatively, said inductive heating element is coated onto at least a part of the perforator. In an alternative embodiment, the inductive heating coil is arranged onto the perforator and the heating element can be arranged as part of the inhalation device. The inductive heating element can be fabricated using various materials as known to a person skilled in the art. Preferably, the heating element has electrically conductive material associated with it. For example, the heating element can be made of metal or it can be made of a non-metallic material and have a metal coating on it. A flux concentrator can optionally be used to increase the flux concentration in the heating element and thereby heat the perforator at a faster rate.

In an embodiment, the inductive heating coils can be provided as part of the inhalation device. The inductive heating coils can be wound around a coil bobbin in various configurations such that upon docking of the piercing device, the perforator including inductive heating element as part thereof can be located at the centre of the induction heating coil. The inductive heating coils can be wound around a coil bobbin in various configurations in order to operate under different design conditions. For example, the density of the inductive heating coils on the bottom side of the coil bobbin can be increased and decreased on the top side in order to create a greater concentration of flux at the bottom side than on the top and thus greater heating ability on the apex portion of the perforator. Likewise, for an opposite effect, the density of coils at the top side could be greater than at the bottom side.

A power circuit can be provided as part of the piercing device or as part of the inhalation device and preferably, as part of the inhalation device for powering the inductive heating mechanism. The power circuit can be a FET power bridge which is just one example of various types of circuits that may be used to power the inductive heating mechanism of the present invention. The power supply can be a DC source and a convertor can provide an AC current through the coil. This can be accomplished by use of the switches and forcing current to flow in opposite directions to simulate AC current. For example, switches can be turned on so that current flows through the resonant capacitor, through the coil, back through the second resonant capacitor and through switch. When one set of switches are open and others are closed, current flows in the opposite direction. Thus, a simulation of AC current can be obtained. Alternatively, a solenoid inductive heating mechanism can also be used with the power circuit. The power source can be sized to provide sufficient power for heating elements that heat the contents of the reservoir. The power source is preferably replaceable and rechargeable and may include devices such as a capacitor, or more preferably, a battery. In a presently preferred embodiment, the power source is a replaceable, rechargeable battery such as nickel cadmium battery cells connected in series as part of the inhalation device. The characteristics required of the power source can be selected in view of the characteristics of other components in the inhalation device and the characteristics of the heating elements.

In order for the inductive heating mechanism to heat the content of the reservoir, a current is passed through one or more inductive heating coils which produces an electromagnetic flux. A heating element is located such that the flux produces eddy currents inside the heating element which in turn heats the heating element. This heat is then transferred to contents of the reservoir by way of direct or indirect thermal conduction.

Although, few embodiments of the present disclosure are described with reference to utilization of inductive heating means/mechanisms, utilization of any other forms/means of heating is completely within the scope of the present invention. The heating mechanism can be a ceramic heater, Kapton heater, laser, induction heater, and/or convection heater. In operation, the heating mechanism (alternatively and synonymously termed as “heater”) increases the temperature of the chamber holding said reservoir and/or the perforator to a predetermined value between 100 to about 230 degrees Celsius or preferably between 120 degrees Celsius to about 220 degrees Celsius. The heater can be adapted to achieve the desired temperature either instantaneously or over a predetermined period of time (e.g. in several stages). A staged temperature change can ensure that certain substances (e.g. cannabinoids) are effectively vaporized as different substances may vaporize at different temperatures. In an embodiment, the chamber can be insulated by any means known to persons skilled in the art to maintain the temperature of the chamber, so that the inhalation device may be handled by the user, and to insulate the electronics section and/or other electronic components of the inhalation device. Alternatively, the outer casing of the inhalation device may be made of an insulation material.

In an embodiment, said at least one perforator is of pyramid shape defining one or a plurality of lateral surfaces and an apex. The pyramid shape may effect ease of penetration of said perforator through the packaging material of the reservoir. In case the reservoir does not include such a packaging and/or if the packaging is removed beforehand and then the reservoir is inserted into said inhalation device, the pyramid shape may assist the perforator to easily sail through/traverse the contents of the reservoir (for example in case the reservoir is filled with solid/powdery substance) to reach to the desired depth of the content/material of the reservoir to ensure uniform heating thereof. In an embodiment, said plurality of passages are defined at any or a combination of at least one of said one or a plurality of lateral surface and said apex. In an embodiment, any or a combination of at least a part of said one or a plurality of lateral surfaces and said apex comes in contact with the content of the reservoir upon docking of said piercing device with the inhalation device. In an embodiment, said at least one perforator defines a plurality of elevations along said one or a plurality of lateral surfaces defining a plurality of peaks and troughs. In an embodiment, at least one of said plurality of passages is defined along a surface of said plurality of elevations. In an embodiment, the passages defined at peaks allow passage of ambient air to the content of the reservoir and the passages defined at troughs allow passage of vapor generated from the content of the reservoir to the mouthpiece to be delivered to the user. In an alternative embodiment, the passages defined at troughs allow passage of ambient air to the content of the reservoir and the passages defined at peaks allow passage of vapor to the mouthpiece to be delivered to the user.

In an embodiment, said at least one perforator is of cylindrical shape with said plurality of passages defined at any or a combination of periphery of the at least one perforator and centre of the at least one perforator. In an embodiment, any or a combination of at least a part of said periphery of the at least one perforator and the centre of said at least one perforator comes in contact with the content of the reservoir upon docking of said piercing device with the inhalation device.

In an embodiment, said at least one perforator is of pyramid shape defining one or a plurality of lateral surfaces and a cylindrical protrusion at apex of said at least one perforator. In an embodiment, said plurality of passages are defined at: (a) any or a combination of periphery of said cylindrical protrusion and centre of said cylindrical protrusion and (b) said one or a plurality of lateral surfaces of said at least one perforator. In an embodiment, said at least one perforator defines a plurality of elevations along said one or a plurality of lateral surfaces defining a plurality of peaks and troughs. In an embodiment, at least one of said plurality of passages is defined at said plurality of troughs. In an embodiment, at least one of said plurality of passages is defined at said plurality of peaks. In an embodiment, the passages defined at peaks allow passage of ambient air to the content of the reservoir and the passages defined at troughs allow passage of vapor generated from the content of the reservoir to the mouthpiece to be delivered to the user. In an alternative embodiment, the passages defined at troughs allow passage of ambient air to the content of the reservoir and the passages defined at peaks allow passage of vapor to the mouthpiece to be delivered to the user. In an embodiment, the passage(s) defined at centre of the cylindrical protrusion define(s) passage for ambient air to enter in contact with at least a part of the content of the reservoir. These advantageous configurations are advantageous in maximizing the amount and uniformity of vapors that can be drawn through the passages to be delivered to a user. Alternatively, the perforator can be of any other geometry, as known to a person skilled in the art, to serve its intended purpose as laid down in various embodiments of the present disclosure. Further, passages can be arranged in any other configuration relative to the perforator, as known to a person skilled in the art, so as to maximize the passage of ambient air reaching to the contents of the reservoir and/or vapors generated from contents of the reservoir to the mouth piece.

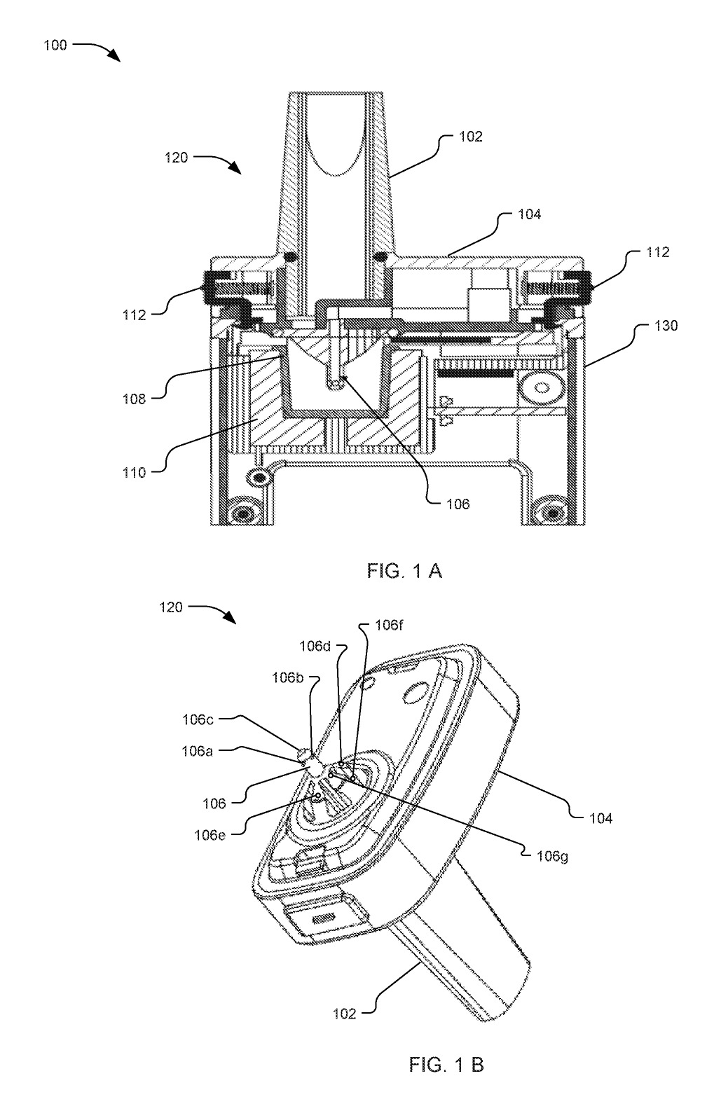

FIG. 1A illustrates an exemplary partially exposed front view 100 of a piercing device 120 docked with an inhalation device 130 in accordance with embodiments of the present disclosure. As illustrated, the piercing device 120 includes a mouth piece 102, a connector 104 and a perforator 106. The perforator 106 includes at least one heating mechanism (not shown). The connector 104 includes a spring biased clip as a locking mechanism, as shown as 112, to detachably connect the piercing device 120 with the inhalation device 130. The inhalation device 130 includes a chamber 108 to hold the reservoir therein. The walls of the chamber 108 can serve as a coil bobbin with inductive heating coil 110 wound thereon. The inhalation device can further include a suitable power circuitry (not shown) to provide sufficient power to the induction heating mechanism.

FIG. 1B illustrates an exemplary perspective view of a piercing device in accordance with embodiments of the present disclosure. The piercing device includes a perforator 106 of pyramid shape, that defines a lateral surface (being conical) with a plurality of elevations defining a plurality of peaks and troughs, with a cylindrical protrusion at apex of the perforator 106. As can be seen, plurality of passages are defined, as shown as 106a, 106b and 106c, at periphery and centre of said cylindrical protrusion. The passage 106c is defined at centre while passages 106a and 106b are defined along periphery of the perforator. The mouth piece 102 defines a chamber connected to passages 106a and 106b to allow passage of the vapors generated from the content of the reservoir to the mouth piece 102. As can also be seen, plurality of passages are defined, as shown as 106d, 106e and 106f and 106g, at peaks and troughs. The passages 106d and 106f are defined at peaks and passages 106e and 106g are defined at troughs. The chamber defined by the mouth piece 102 is connected to passages 106e and 106g to allow passage of the vapors generated from the content of the reservoir to the mouth piece 102, while the passages 106d and 106f allow passage of ambient air to the content of the reservoir.

The provision of plurality of passages with specific configurations embodied herein forming a part of the perforator 106 ensures maximum passage of ambient air through the contents of the reservoir and maximum passage of vapors generated from contents of the reservoir to the mouth piece. Further, the advantageous piercing device can easily rupture the packaging material of the reservoir to access contents thereof. In case the reservoir does not include such a packaging and/or if the packaging is removed beforehand and then the reservoir is inserted into said inhalation device, the perforator can easily access contents of the reservoir by sailing through/traversing the superficial layers/contents of the reservoir (for example in case the reservoir is filled with solid/powdery substance) to reach to the desired depth of the content/material of the reservoir to ensure uniform heating thereof. The piercing device, in accordance with embodiments of the present disclosure, can operate in the temperature ranging up to about 300° C., can pierce aluminium foil with thickness of about 20 mm and can effectively work with a induction heating coil to heat the content of the reservoir up to 210° C.

In operation, a reservoir (not shown) is placed into a space defined by walls of the chamber 108 of the inhalation device 130. The piercing device 120 is then docked with the inhalation device 130 so that the perforator 106 penetrates the packaging material of the reservoir accessing contents of the reservoir. The locking mechanism 112 (shown as spring biased clip) ensures detachable coupling of the piercing device 120 with the inhalation device 130. Penetration of the perforator enables passage of ambient air to at least a part of the content of the reservoir through the passage 106c. Upon activation of inductive heating mechanism, electrical current is sent through the inductive heating coils creating a magnetic field. The flux from this magnetic field is coupled in the perforator. As the flux enters the perforator, eddy currents created in the electrically conductive element (inductive heating element) heat the perforator which then transfers that heat to the contents of the reservoir. The inductive heating of the heating element heats the content of the reservoir to a desirable temperature range in a rapid manner allowing generation of vapors therefrom. The vapors then pass through the passages 106a, 106b, 106e and 106g to enter mouth piece 102 to be inhaled by the user.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable people having ordinary skill in the art to make and use the invention when combined with information and knowledge available to the person having ordinary skill in the art.

### ADVANTAGES OF THE PRESENT DISCLOSURE

The present disclosure provides a piercing device for inhalation device that overcomes one or more limitations associated with conventional devices.

The present disclosure provides a piercing device that allows easy penetration of the packaging material of the reservoir allowing access to the contents thereof.

The present disclosure provides a piercing device that allows passage of ambient air and vaporized contents of the reservoir without clogging.

The present disclosure provides a piercing device that is easy to use.

I/we claim:

1. A piercing device for use with an inhalation device, the piercing device comprising:
at least one perforator;
a plurality of passages defined along said at least one perforator; and
at least one heating mechanism incorporated in said at least one perforator,
wherein docking of said piercing device with the inhalation device holding a reservoir allows said at least one perforator to access content of the reservoir, and wherein activation of said at least one heating mechanism allows generation of vapors from the content of the reservoir to be carried through at least one of said plurality of passages to a user.

2. The piercing device as claimed in claim 1, wherein said piercing device further comprises a mouth piece and a connector.

3. The piercing device as claimed in claim 2, wherein said connector defines a first chamber connected to said at least one of the plurality of passages to allow passage of the vapors generated from the content of the reservoir through said at least one of the plurality of passages.

4. The piercing device as claimed in claim 2, wherein said mouth piece defines a second chamber connected to said first chamber to allow passage of the vapors from said first chamber to said user.

5. The piercing device as claimed in claim 2, wherein said connector and said mouth piece define a contiguous chamber connected to said at least one of the plurality of passages to allow passage of the vapors generated from the content of the reservoir to the user.

6. The piercing device as claimed in claim 1, wherein said connector comprises at least one locking mechanism to detachably connect said perforator with said inhalation device.

7. The piercing device as claimed in claim 1, wherein said at least one heating mechanism comprises an induction heating mechanism.

8. The piercing device as claimed in claim 7, wherein said induction heating mechanism is selected from a group consisting of an inductive heating element and an inductive heating coil.

9. The piercing device as claimed in claim 8, wherein the inductive heating element is dispersed in said at least one perforator and wherein the inductive heating coil is arranged as part of the inhalation device.

10. The piercing device as claimed in claim 1, wherein said at least one perforator is of pyramid shape defining one or a plurality of lateral surfaces and an apex.

11. The piercing device as claimed in claim 10, wherein said plurality of passages are defined at any or a combination of at least one of said one or a plurality of lateral surface and said apex, further wherein any or a combination of at least a part of said one or a plurality of lateral surfaces and said apex comes in contact with the content of the reservoir upon docking of said piercing device with the inhalation device.

12. The piercing device as claimed in claim 11, wherein said at least one perforator defines a plurality of elevations along said one or a plurality of lateral surfaces defining a plurality of peaks and troughs, further wherein at least one of said plurality of passages is defined along a surface of said plurality of elevations.

13. The piercing device as claimed in claim 1, wherein said at least one perforator is of cylindrical shape with said plurality of passages defined at any or a combination of periphery of the at least one perforator and centre of the at least one perforator.

14. The piercing device as claimed in claim 13, wherein any or a combination of at least a part of said periphery of the at least one perforator and the centre of said at least one perforator comes in contact with the content of the reservoir upon docking of said piercing device with the inhalation device.

15. The piercing device as claimed in claim 1, wherein said at least one perforator is of pyramid shape defining one or a plurality of lateral surfaces and a cylindrical protrusion at apex of said at least one perforator.

16. The piercing device as claimed in claim 15, wherein said plurality of passages are defined at: (a) any or a combination of periphery of said cylindrical protrusion and centre of said cylindrical protrusion and (b) said one or a plurality of lateral surfaces of said at least one perforator.

17. The piercing device as claimed in claim 15, wherein said at least one perforator defines a plurality of elevations along said one or a plurality of lateral surfaces defining a plurality of peaks and troughs.

18. The piercing device as claimed in claim 17, wherein at least one of said plurality of passages is defined at said plurality of troughs.

19. The piercing device as claimed in claim 17, wherein at least one of said plurality of passages is defined at said plurality of peaks.

20. The piercing device as claimed in claim 17, wherein said at least one of said plurality of passages defined at said plurality of peaks allows passage of ambient air to the content of the reservoir, and wherein said at least one of said plurality of passages defined at said plurality of troughs allows passage of vapors generated from the content of the reservoir to the mouth piece.
